(19) United States
(12) Patent Application Publication
(10) Pub. No.: US 2021/0270855 A1
(43) Pub. Date: Sep. 2, 2021

(54) PIP2 AS A MARKER FOR HDL FUNCTION AND CARDIOVASCULAR DISEASE RISK

(71) Applicant: The Cleveland Clinic Foundation, Cleveland, OH (US)

(72) Inventors: Jonathan D. Smith, Shaker Heights, OH (US); Kailash Gulshan, Mayfield Heights, OH (US); Stanley L. Hazen, Pepper Pike, OH (US)

(21) Appl. No.: 17/203,883

(22) Filed: Mar. 17, 2021

Related U.S. Application Data
(63) Continuation of application No. 16/544,356, filed on Aug. 19, 2019, now abandoned.
Application No. 16/544,356 is a continuation of application No. 15/598,522, filed on May 18, 2017, now abandoned.
(60) Provisional application No. 62/337,952, filed on May 18, 2016.

(51) Int. Cl. G01N 33/92; C12N 15/113; G16H 50/30; G01N 33/68; A61K 45/06; C07K 16/40

(52) U.S. Cl. CPC G01N 33/92; C12N 15/1138; C12Y 301/03078; G16H 50/30; G16H 15/00; A61K 45/06; C07K 16/40; C12N 15/1137; G01N 33/6893

(57) ABSTRACT

Provided herein are compositions, systems, kits, and methods for detecting cardiovascular disease, risk of cardiovascular disease, and/or reverse cholesterol transport potential in a subject based on the levels of PIP2 phospholipid in the subject.

The present application is a continuation of U.S. patent application Ser. No. 16/544,356, filed Aug. 19, 2019, which is a continuation of U.S. patent application Ser. No. 15/598,522, filed May 18, 2017, now abandoned, which claims priority to U.S. Provisional Application Ser. No. 62/337,952, filed May 18, 2016, each of which is herein incorporated by reference in its entirety.

### STATEMENT REGARDING FEDERAL FUNDING

This invention was made with government support under grant numbers HL098055 and HL128268 awarded by the National Institutes of Health. The government has certain rights in the invention.

### FIELD

Provided herein are compositions, systems, kits, and methods for detecting cardiovascular disease, risk of cardiovascular disease, and/or reverse cholesterol transport potential in a subject based on the levels of phosphatidylinositol (4,5) bis-phosphate, herein abbreviated as PIP2, a phospholipid in the subject.

### BACKGROUND

HDL plays a role in many cellular pathways via diverse mechanisms, including anti-thrombotic, vasoprotective, anti-inflammatory, and cholesterol efflux activities. HDL assembly involves the cellular lipidation of extracellular apolipoprotein A-I (apoA1) by the membrane protein ABCA1. The importance of the ABCA1 pathway in generating nascent HDL (nHDL) is demonstrated in human patients carrying mutations in ABCA1 (Tangier disease) who have extremely low levels of plasma HDL. These patients have increased accumulation of cholesterol in peripheral tissues, resulting in premature atherosclerotic vascular disease. Although recent trials of HDL-cholesterol (HDL-C) raising drugs have not appeared to prevent cardiovascular events, a consensus is building that it is HDL function in reverse cholesterol transport (RCT), rather than the levels of HDL-C, that is protective against cardiovascular disease. For example, cholesterol efflux capacity of apoB-depleted serum is inversely associated with both prevalent and incident cardiovascular disease, independent of HDL-C levels.

The mechanism of cellular lipidation of apoA1 by ABCA1 is not understood at the molecular level with various models discussed in recent reviews. ABCA1 has two well-established intermediate activities leading to apoA1 lipidation: 1) the outward translocation or “flopping” of PS to cell surface, and 2) apoA1 binding to the cell surface. We recently characterized a third activity, the unfolding of N-terminal hairpin of apoA1 on the cell surface. Interestingly, apoA1 binding to the cell surface is independent of the PS floppase activity of ABCA1, as the W590S-ABCA1 Tangier disease mutation is defective in PS floppase but not in apoA1 binding, while the C1477R-ABCA1 Tangier disease mutant is defective in apoA1 binding but not in PS floppase activity. It is important to note that both W590S and C1477R have impaired apoA1 lipidation, indicating that PS floppase and apoA1 cell surface binding are both required for efficient transfer of cellular lipids to apoA1 during nHDL biogenesis.

Several models have been proposed to explain the mechanism responsible for the specific binding of apoA1 to ABCA1-expressing cells: a) apoA1 binding to cell surface phosphatidylserine (PS) due to ABCA1 PS floppase activity; b) direct interaction between apoA1 and ABCA1 as demonstrated by protein cross-linking; c) low-capacity binding of apoA1 to ABCA1 and high-capacity binding of apoA1 to membrane lipids; d) apoA1 interaction with membrane protrusions due to ABCA1 bulk phospholipid outward translocase (floppase) activity. Recent solid-phase binding studies from the Molday lab showed no direct binding between apoA1 and purified ABCA1 in the presence or absence of several classes of phospholipids including PS. Since these experiments were carried using immobilized ABCA1, the possibility of apoA1 and ABCA1 direct interaction on cell surface cannot be ruled out.

The major phospholipid constituents of HDL are phosphatidylcholine (PC), PS, phosphatidylethanolamine (PE), and phosphatidylinositol (PI). Unlike other structural phospholipids, phosphatidylinositol phosphates (PIPs) are minor components of cellular membranes, but they serve as critical integral signaling molecules for multiple pathways. PI(4,5)bis-phosphate (PIP2) is the major cellular PIP species and it is predominantly found on the inner leaflet of the plasma membrane where it play roles in many cellular processes such as membrane ruffling, endocytosis, exocytosis, protein trafficking and receptor mediated signaling. The PIP2 binds to various effector proteins through interacting with pleckstrin homology (PH) domains thereby regulating the effector protein cellular localization and activity. PIP2 synthesis is tightly regulated by P1-kinases, such as PI4P-5 kinase, and PIP phosphatases, such as PTEN.

### SUMMARY

Provided herein are compositions, systems, kits, and methods for detecting cardiovascular disease, risk of cardiovascular disease, and/or reverse cholesterol transport potential in a subject based on the levels of phosphatidylinositol (4,5) bis-phosphate, herein abbreviated as PIP2, a phospholipid in the subject.

In some embodiments, provided herein are methods for using circulating PIP2 phospholipid as a marker of HDL function and a diagnostic for major adverse cardiovascular events. It was discovered that phosphatidylinositol (4.5) bis-phosphate, hereafter called PIP2, plays an essential role in HDL biogenesis, and that it is carried in the circulation on HDL in both humans and mice. Furthermore, PIP2 carried on HDL can be delivered to target cells, which is in part mediated by the HDL receptor SR-BI. Based on these discoveries, in certain embodiments, the circulating levels of PIP2 can be measured (e.g., using a commercial ELISA assay) and such levels used as: 1) a surrogate for HDL function in reverse cholesterol transport; 2) An indicator of the cholesterol acceptor activity of HDL; 3) a diagnostic to predict risk for future major adverse cardiovascular events, such as myocardial infarction, stroke, the need for revascularization, and coronary or cerebral sudden death; 4) an indicator for drug treatment and measure of drug efficacy.

In some embodiments, provided herein are methods for performing an activity based on concentration level of PIP2 in a biological sample from a subject comprising: a) determining the concentration level (e.g., μg/ml or μM) of total PIP2 in a biological sample from a subject, and/or determining the concentration level (e.g., μg/ml or μM) of HDL-associated PIP2 in the biological sample from the subject; and b) performing at least one of the following: i) identifying decreased (e.g., compared to control levels from disease free or general population) total or HDL-associated PIP2 levels in the biological sample, and treating the subject with a CVD therapeutic agent; ii) generating and/or transmitting a report that indicates the total or HDL-associated PIP2 levels are decreased (e.g., compared to control levels from disease free or general population) in the sample, and that the subject is in need of a CVD therapeutic agent; iii) generating and/or transmitting a report that indicates the total or HDL-associated PIP2 levels are decreased (e.g., compared to control levels from disease free or general population) in the sample, and that the subject has or is at risk of cardiovascular disease (e.g., atherosclerotic CVD) or complication of cardiovascular disease; iv) generating and/or transmitting a report that indicates the total or HDL-associated PIP2 levels are elevated (e.g., compared to control levels from disease free or general population) in the sample, and that the subject has increased reverse-cholesterol transport function; and v) characterizing the subject as having CVD or having an increased risk for having or developing CVD (e.g., atherosclerotic disease).

In certain embodiments, the CVD therapeutic agent is selected from the group consisting of: an antibiotic, a statin, a probiotic, an alpha-adrenergic blocking drug, an angiotensin-converting enzyme inhibitor, an angiotensin receptor antagonist, an antiarrhythmic drug, an anticoagulant, an antiplatelet drug, a thromybolytic drug, a beta-adrenergic blocking drug, a calcium channel blocker, a brain acting drug, a cholesterol-lowering drug, a TMEM55b inhibitor, a OCRL1 inhibitor, a digitalis drug, a diuretic, a nitrate, a peripheral adrenergic antagonist, and a vasodilator. In particular embodiments, the subject is a human. In other embodiments, the biological sample is a plasma, serum, blood, urine, or similar sample.

In further embodiments, the biological sample is treated to isolate HDL particles, and treating the HDL sample or the unfractionated sample with solvents to extract PIP2 away from proteins in the HDL of unfractionated sample. In other embodiments, the biological sample is treated with ultracentrifugation or apoB precipitation reagent to generate the HDL sample, wherein the HDL sample is free of detectable LDL, IDL, and VLDL. In additional embodiments, the HDL sample or the unfractionated sample is treated with weak detergents to cause PIP2 to dissociate away from HDL or sample proteins.

In certain embodiments, the cardiovascular disease or complication of cardiovascular disease is one or more of the following: non-fatal myocardial infarction, stroke, angina pectoris, transient ischemic attacks, congestive heart failure, aortic aneurysm, aortic dissection, and death. In other embodiments, the risk of cardiovascular disease is a risk of having or developing cardiovascular disease within the ensuing three years.

In some embodiments, provided herein are systems comprising: a) a report for a subject indicating that the subject has decreased total or HDL-associated PIP2 levels; and b) a CVD therapeutic agent.

In certain embodiments, provided herein are methods comprising: a) identifying a subject as having reduced levels of PIP2, and b) treating the subject with a CVD therapeutic agent. In further embodiments, the identifying comprises receiving the report.

In some embodiments, provided herein are methods for evaluating the effect of a cardiovascular disease (CVD) therapeutic agent on a subject comprising: a) determining a first level (e.g., concentration) of PIP2 in a bodily sample (e.g., plasma) taken from a subject (e.g., human subject) prior to administration of a CVD therapeutic agent (e.g., lipid lowering agent), and b) determining a second level of PIP2 in a corresponding bodily fluid taken from the subject following administration of the CVD therapeutic agent.

In certain embodiments, an increase in the first level to the second level is indicative of a positive effect of the CVD therapeutic agent on cardiovascular disease in the subject. In further embodiments, the CVD therapeutic agent comprises a lipid reducing agent (e.g., a statin). In further embodiments, the CVD therapeutic agent is selected from the group consisting of: an anti-inflammatory agent, a TMEM55b inhibitor, a OCRL1 inhibitor, an insulin sensitizing agent, an anti-hypertensive agent, an anti-thrombotic agent, an anti-platelet agent, a fibrinolytic agent, a direct thrombin inhibitor, an ACAT inhibitor, a CETP inhibitor, and a glycoprotein IIb/IIIa receptor inhibitor. In particular embodiments, the CVD is atherosclerotic CVD. In other embodiments, the subject has been diagnosed as having CVD. In further embodiments, the subject has been diagnosed as being at risk of developing CVD. In certain embodiments, the bodily sample is a plasma, blood, serum, urine, or other sample. In additional embodiments, the determining in step a) and/or step b) comprises contacting the bodily sample with an anti-PIP2 antibody (e.g., ELISA or immunoturbometric assay). In other embodiments, the determining in step a) and/or step b) further comprises spectrophotometrically detecting the anti-PIP2 antibody. In certain embodiments, the anti-PIP2 antibody is a monoclonal antibody (e.g., anti-PIP2 antibody 2C11 from Abcam, Cambridge, Mass.).

In certain embodiments, provided here are methods comprising: administering a transmembrane protein 55B (Tmem55b) inhibitor and/or an inositol polyphosphate-5-phosphatase (OCRL1) inhibitor to a subject, wherein said subject has, or is suspected of having, cardiovascular disease (e.g., atherosclerotic disease).

In particular embodiments, the Tmem55b inhibitor comprises a Tmem55b siRNA sequence (e.g., SEQ ID NOS:1-3), a Tmem55b antisense sequence, a small molecule, and/or an anti-Tmem55b antibody or antigen binding fragment thereof (e.g., monoclonal antibody or antigen binding portion thereof). In further embodiments, the OCRL1 inhibitor comprises an OCLR1 siRNA sequence (e.g., SEQ ID NOS:4-6), an OCRL1 antisense sequence, a small molecule (e.g., YU142717, YU144805, or YU1422670), and/or an anti-OCRL1 antibody or antigen binding fragment thereof (e.g., monoclonal antibody or antigen binding portion thereof). In certain embodiments, Tmem55b inhibitor and/or said OCLR1 inhibitor is administered at a level to increase the PIP2 levels in said subject at least 10% (e.g., at least 10% . . . 20% . . . 30% . . . 40% . . . 50% . . . 75% . . . or 200%).

### DESCRIPTION OF THE FIGURES

FIGS. 1A-H. ApoA1 binds PIP2. A. Lipid-protein overlay assay using PIP strip for detection of apoA1 binding to cellular lipids. B. SPR assay showing direct binding of apoA1 (550 nM) (blue line) to biotinylated PIP2 immobilized on an SPR sensor chip (green and red lines are buffer controls). C. SPR assay showing PIP2 binding (top two lines) to immobilized biotinylated apoA1, while PC showed no binding (line labeled 1 μM PC). D. Fluorescent anisotropy of bodipy-lableled PIP2 binding to varying concentrations of apoA1. E. SPR analysis showing binding of full-length and truncation mutants of apoA1 to immobilized PIP2. F. Liposome floatation assay showing increased apoA1 floatation with POPC MLVs containing 5 mol % PIP2 vs. those without PIP2. G. DMPC clearance assay using MLVs with or w/o 5 mole % PIP2 incubated with apoA1 (100:1 lipid:apoA1 mole ratio) at 25° C. with lipid solubilization determined by measuring turbidity at 325 nm. H. Lipid-free apoA1 was incubated with or without PIP2 or palmitoyloleoyl-phophatidylserine (POPS) and subjected to BS3 mediated cross linking followed by SDS-PAGE and apoA1 western-blot to assess apoA1 monomer-oligomer confirmations.

FIGS. 2A-G. ABCA1 flops PIP2 promoting apoA1 binding and cholesterol efflux. A. Cell surface PIP2 assessed by flow cytometry in RAW264.7 cells±ABCA1 induction that were pretreated±PI-PLC (left panel; RFI, relative fluorescence intensity; mean±SD; different letters show p<0.001 by ANOVA Bonferroni posttest, n=3). Western blot of RAW264.7 cell extracts showing expression of ABCA1±PI-PLC treatment (right panel). B. ABCA1 expression redistributed the PIP2 reporter, PH-PLCδ-eGFP, away from the plasma membrane in stably transfected RAW264.7 cells. C. ApoA1 binding to RAW264.7 cells±ABCA1 expression and PI-PLC treatment assessed by flow cytometry (mean±SD; different letters show p<0.01, by ANOVA Bonferroni posttest, n=3). D. % Cholesterol efflux from RAW264.7 cells±ABCA1 expression chased with apoA1 (5 μg/ml)±2.5 units/ml of PI-PLC (mean±SD; different letters show p<0.01, by ANOVA Bonferroni posttest, n=3). E. ApoA1 binding to ABCA1 expressing RAW264.7 cells±exogenous PIP2 (PIP2:apoA1, 5:1 mole ratio) or PIP2 specific monoclonal antibody (2 μg/ml) (mean±SD; different letters show p<0.01, by ANOVA Bonferroni posttest, n=3). F. Cholesterol efflux from ABCA1-expressing RAW264.7 cells to apoA1 (5 μg/ml) that was pre-incubated±exogenous PIP2 (PIP2:apoA1, 5:1 mole ratio; mean±SD; ***, p<0.001 by 2-tailed t-test, n=3). g, Cholesterol efflux to apoA1, cell surface PIP2, cell surface PS, and apoA1 cellular binding were measured in control HEK cells and those stably transfected with either ABCA1, W590S-ABCA1, or C1477R-ABCA1 isoforms. Loss of cell surface PIP2 and apoA1 binding were observed for the C1447R isoform, while loss of cell surface PS was observed for the W590S isoform (mean±SD; different letters show p<0.001 by ANOVA Bonferroni posttest, n=3).

FIG. 3. Modulation of PIP metabolism regulates cholesterol efflux. Panel A and Panel B—Cholesterol efflux from ABCA1 induced RAW264.7 cells to apoA1 after PIP2 lowering treatments with 1 μM PI4K inhibitor PIK-93 (a) or 1 μM PTEN inhibitor SF 1670 (b) (mean±SD; p<0.001, different letters show p<0.001 by ANOVA Bonferroni posttest, n=3). Panel C. siRNA mediated knockdown of PIP2 phosphatase Tmem55b observed by western blot (left) and its effect on cholesterol efflux (right, mean±SD; ***, p<0.001, by two-tailed t-test, n=3).

FIG. 4. PIP2 circulates on plasma HDL. Panel A. ABCA1 mediates efflux of [³H]inositol labeled lipids to apoA1 from RAW264.7 cells (mean±SD; ***, p<0.001, by two-tailed t-test, n=3). Panel B. PIP2 and PI4P in lipids from RAW264.7 cells and in apoA1-containing conditioned media visualized by lipid-protein overlay assays using tagged PIP2 or PI4P binding proteins. Panel C. ABCA1 dependent efflux of PIP2 to apoA1 in conditioned media assessed by ELISA, normalized to cell protein (mean±SD; ***, p<0.001, by two-tailed t-test, n=3). Panel D. PIP2 (ELISA assay, blue bars) and cholesterol (open bars) levels in plasma derived from apoA1 KO, WT, and apoA1 transgenic mice (mean±SD). Panel E. Plasma PIP2 radioactivity in apoA1 KO and WT recipients 3 d after s.c. implantation of bone marrow macrophages labeled with [3H]myo-inositol (mean±SD; **, p<0.01, by two-tailed t-test, n=3). Panel F. PIP2 (ELISA assay, blue circles) and cholesterol (open circles) levels in human plasma separated by FPLC. Panel G. Human HDL analyzed by liquid chromatography mass spectrometry to identify endogenous PIP2 fatty acid species. Panel H. Uptake of [³H]PIP2 labeled HDL by BHK cells±SR-BI expression (mean±SD; **, p<0.01, by two-tailed t-test, n=3).

FIGS. 5A-E. PIP2 interaction with HDL apolipoproteins. A. Lipid-protein overlay assay using spying strip demonstrates that apoA1 does not bind appreciably to various cellular lipids including PC, sphingomyelin, cholesterol, and sphingosine-1-phosphate. B. SPR assay showing dose-dependent direct binding PIP2 to immobilized apoA1. C. SPR assay showing dose-dependent direct binding PIP2 to immobilized apoA2. D. SPR assay showing dose-dependent direct binding PIP2 to immobilized apoE. E. Liposome clearance assay using MLVs made of PC:POPS:Cholesterol (70:20:10 mole ratio) with or without 5 mole % PIP2 incubated with apoA1 (100:1, lipid:apoA1 mole ratio) at 25° C., pH 5.0. Lipid solubilization determined by measuring turbidity at 325 nm.

FIG. 6. ABCA1 flops PIP2 promoting apoA1 binding and cholesterol efflux in additional cell lines. Panel A. Cell surface PIP2 assessed by flow cytometry in BHK±ABCA1 induction that were pretreated±PI-PLC (RFI, relative fluorescence intensity; mean±SD; different letters show p<0.001 by ANOVA Bonferroni posttest, n=3). Panel B. Western blot of BHK cell extracts showing expression of ABCA1±PI-PLC treatment. C Panel. ABCA1 expression redistributed the PIP2 reporter, PH-PLCδ-eGFP, away from the plasma membrane in stably transfected BHK cells. Panel D. ApoA1 binding to HEK293 cells and ABCA1 stably transfected cells±PI-PLC treatment assessed by flow cytometry (mean±SD; different letters show p<0.001, by ANOVA Bonferroni posttest, n=3). Panel E. % Cholesterol efflux from BHK cells±ABCA1 expression chased with apoA1 (5 μg/ml)±2.5 units/ml of PI-PLC (mean±SD; different letters show p<0.001, by ANOVA Bonferroni posttest, n=3).

FIG. 7. Schematic diagram showing PIP2 metabolism. PIP2 can be generated from PI4P or PIP3 via PI4P 5 kinase (PI5K) and PTEN, respectively. Inhibitors to these two enzymes were used to decrease cellular PIP2 levels in FIG. 7. PIP2 can be dephosphorylated to PI5P by the PIP2 phosphatase TMEM55B. Knockdown of Tmem55b was used to increase cellular PIP2 levels in FIG. 7.

FIG. 8. PIP2 is effluxed from BHK cells via ABCA1 to apoA1. Panel A. ABCA1 mediates efflux of [³H]inositol labeled lipids to apoA1 from BHK cells (mean±SD; different letters show p<0.001 by ANOVA Bonferroni posttest, n=3). Panel B. PIP2 in apoA1 containing conditioned media from BHK cells with or without ABCA1 expression. PIP2 was visualized by spotting extracted media lipids onto a membrane followed by protein overlay with the tagged PIP2 binding protein GST-PLCδ-PH.

FIG. 9. Hypothetical model for ABCA1 mediated HDL biogenesis. While the present invention is not limited to any particular mechanism, and an understanding of the mechanism is not necessary to practice the invention, it is believed, based on this model, that PS and PIP2 floppase activities of ABCA1 remodel the plasma membrane and are independent of each other, with the latter mediating apoA1 binding. After binding to cell surface PIP2, apoA1 monomers insert into the membrane where 2 or 3 apoA1 molecules can assemble into a nascent HDL (nHDL) that is released from the cell surface. Both PS and PIP2 floppase activities are required for efficient apoA1 lipidation and nHDL release.

### DEFINITIONS

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

As used herein, the term “atherosclerotic cardiovascular disease” or “disorder” refers to a subset of cardiovascular disease that include atherosclerosis as a component or precursor to the particular type of cardiovascular disease and includes, without limitation, CAD, PAD, cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening and/or plaque ruptures. Thus atherosclerotic diseases or disorders include the consequences of atheromatous plaque formation and rupture including, without limitation, stenosis or narrowing of arteries, heart failure, aneurysm formation including aortic aneurysm, aortic dissection, and ischemic events such as myocardial infarction and stroke. In certain embodiments of this disclosure, the subject has atherosclerotic cardiovascular disease.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject.

### DETAILED DESCRIPTION

Provided herein are compositions, systems, kits, and methods for detecting cardiovascular disease, risk of cardiovascular disease, and/or reverse cholesterol transport potential in a subject based on the levels of phosphatidylinositol (4,5) bis-phosphate, herein abbreviated as PIP2, a phospholipid in the subject.

While the present invention is not limited to any particular mechanism, and an understanding of the mechanism is not necessary to practice the invention, work conducted during the development of the present disclosure discovered that: 1) Apolipoprotein A1 (apoA1) binds specifically to PIP2 with a dissociation constant of ˜100 nM; 2) PIP2 on liposomes increases their solubilization by apoA1; 3) ABCA1, the cell membrane protein that generates nascent HDL, transfers PIP2 from the inner to the outer leaflet of the plasma membrane; 4) The ability of ABCA1 to translocate PIP2 to the outer leaflet of the plasma membrane is independent of ABCA1's ability to translocate phosphatidylserine (PS) to the outer leaflet of the plasma membrane; 5) The PIP2 on the outer leaflet of the plasma membrane, due to ABCA1, is responsible and required for the observed binding of apoA1 to ABCA1 expressing cells, as well as for cholesterol efflux to apoA1; 6) PIP2 is effluxed from ABACI expressing cells to apoA1 containing media; 7) The PIP2 levels in mouse blood are dependent upon the expression level of apoA1 and are associated with HDL-C levels. Comparison of plasma PIP2 in the apoA1 knockout and over expressing mice shows that most plasma PIP2 is on HDL and not associated with albumin or other plasma proteins; 8) In human plasma almost all of the circulating PIP2 is associated with HDL, showing that there is not very much exchange of PIP2 onto LDL particles; and 9) PIP2 on HDL can be taken up by target cells, which can be partially mediated by the HDL receptor, SR-B1.

Although HDL-cholesterol (HDL-C) is inversely associated with cardiovascular disease (CVD) in epidemiological studies, recent drug trials and a genetic method call Mendelian randomization have failed to demonstrate that HDL-C is causally protective against CVD. Instead, there is a consensus building that it is HDL function which is causally protective, which is not captured by static measurements of HDL-C. As HDL participates in the reverse cholesterol transport pathway, this is one function of HDL that has been associated with decreased CVD risk, as measured by the cholesterol acceptor activity of apoB-depleted serum using cholesterol labeled cells in culture. This is a cumbersome assay, not easily scaled up. The present disclosure proposes that plasma PIP2 levels serve as a surrogate for HDL's function in reverse cholesterol transport and are useful as a biomarker that be used to predict CVD risk.

In this disclosure, it was demonstrated that PIP2 is associated with human HDL and that one can measure its levels using, for example, a commercially available ELISA assay or other detection methods (e.g., mass spectrometry). In certain embodiments, the present invention may be used as a diagnostic to predict CVD risk, to help select patients for drug therapy, and to determine the efficacy of drug treatments.

In certain embodiments, the CVD therapeutic agent comprises an antibiotic. Examples of such antibiotics include, but are not limited to, a broad spectrum antibiotic, Ampicillin; Bacampicillin; Carbenicillin Indanyl; Mezlocillin; Piperacillin; Ticarcillin; Amoxicillin-Clavulanic Acid; Ampicillin-Sulbactam; Benzylpenicillin; Cloxacillin; Dicloxacillin; Methicillin; Oxacillin; Penicillin G; Penicillin V; Piperacillin Tazobactam; Ticarcillin Clavulanic Acid; Nafcillin; Cephalosporin I Generation; Cefadroxil; Cefazolin; Cephalexin; Cephalothin; Cephapirin; Cephradine; Cefaclor; Cefamandol; Cefonicid; Cefotetan; Cefoxitin; Cefprozil; Ceftmetazole; Cefuroxime; Loracarbef; Cefdinir; Ceftibuten; Cefoperazone; Cefixime; Cefotaxime; Cefpodoxime proxetil; Ceftazidime; Ceftizoxime; Ceftriaxone; Cefepime; Azithromycin; Clarithromycin; Clindamycin; Dirithromycin; Erythromycin; Lincomycin; Troleandomycin; Cinoxacin; Ciprofloxacin; Enoxacin; Gatifloxacin; Grepafloxacin; Levofloxacin; Lomefloxacin; Moxifloxacin; Nalidixic acid; Norfloxacin; Ofloxacin; Sparfloxacin; Trovafloxacin; Oxolinic acid; Gemifloxacin; Pefloxacin; Imipenem-Cilastatin Meropenem; Aztreonam; Amikacin; Gentamicin; Kanamycin; Neomycin; Netilmicin; Streptomycin; Tobramycin; Paromomycin; Teicoplanin; Vancomycin; Demeclocycline; Doxycycline; Methacycline; Minocycline; Oxytetracycline; Tetracycline; Chlortetracycline; Mafenide; Silver Sulfadiazine; Sulfacetamide; Sulfadiazine; Sulfamethoxazole; Sulfasalazine; Sulfisoxazole; Trimethoprim-Sulfamethoxazole; Sulfamethizole; Rifabutin; Rifampin; Rifapentine; Linezolid; Streptogramins; Quinopristin Dalfopristin; Bacitracin; Chloramphenicol; Fosfomycin; Isoniazid; Methenamine; Metronidazol; Mupirocin; Nitrofurantoin; Nitrofurazone; Novobiocin; Polymyxin; Spectinomycin; Trimethoprim; Colistin; Cycloserine; Capreomycin; Ethionamide; Pyrazinamide; Para-aminosalicyclic acid; and Erythromycin ethylsuccinate.

In certain embodiments, an OCRL1 inhibitor is employed to treat cardio vascular disease. The present disclosure is not limited by the type of inhibitor. In certain embodiments, the OCRL1 inhibitor is YU142717, YU144805, or YU142670 as described in Pirruccello et al., ACS Chem Biol. 2014 Jun. 20; 9(6): 1359-1368, which is herein incorporated by reference in its entirety. The structures of YU142717, YU144805, or YU142670 are shown below:

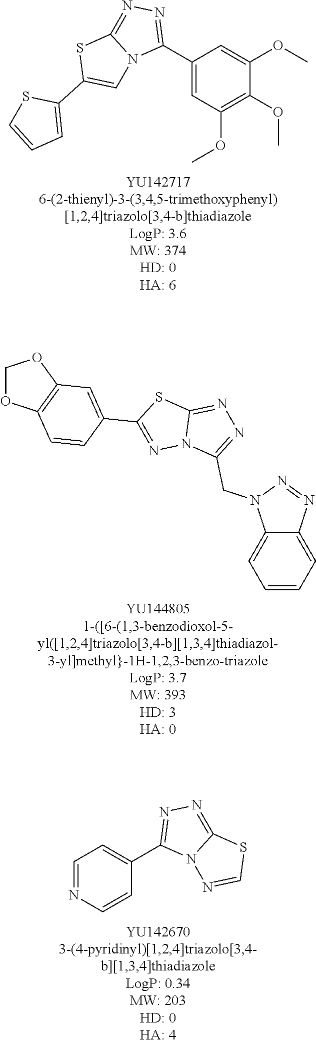 [SMILES: COc1cc(-c2nnc3sc(-c4cccs4)cn23)cc(OC)c1OC ; c1ccc2c(c1)nnn2Cc1nnc2sc(-c3ccc4c(c3)OCO4)nn12 ; c1cc(-c2nnc3scnn23)ccn1]

In other embodiments, the OCRL1 inhibitor comprises an siRNA sequence, such as one selected from SEQ ID NOS:4-6, which are shown below:

Human OCRL siRNA sequences (start is relative to coding sequence start site in mRNA):

| Sequence | Start | GC % | SEQ ID NO: |
|---|---|---|---|
| GAAAGGATCAGTGTCGATACA | 986 | 42.86 | (SEQ ID NO: 4) |
| GAGGCTCTGTGCCGTATGAAA | 2053 | 52.38 | (SEQ ID NO: 5) |
| GTCATCTGTTACGAGCTGTAT | 2380 | 42.86 | (SEQ ID NO: 6). |

In some embodiments, a Tmemb55 inhibitor is employed to treat cardiovascular disease in a subject. In particular embodiments, the Tmem55b inhibitor comprises an siRNA sequence, such as one selected from SEQ ID NOS:1-3, which are shown below:

Human TMEM55B siRNA sequences (start is relative to coding sequence start site in mRNA):

| Sequence | Start | GC % | SEQ ID NO: |
|---|---|---|---|
| GTTCGATGCCCCTGTAACTGT | 367 | 52.38 | (SEQ ID NO: 1) |
| GCAGATACCCACGTAAGAGAT | 614 | 47.62 | (SEQ ID NO: 2) |
| GGCTCTTTATTGGGCCTGTAT | 780 | 47.62 | (SEQ ID NO: 3) |

### EXAMPLES

The following examples are illustrative and not intended to limit the scope of the present invention.

### Example 1

High density lipoprotein (HDL) assembly involves the cellular lipidation of apolipoprotein A-I (apoA1) by the membrane protein ATP cassette binding protein A1 (ABCA1)¹. ABCA1 has two known intermediate activities in HDL biogenesis, the translocation of phosphatidylserine (PS) from the inner to outer leaflet of the cell membrane and the cellular binding of apoA1^{2, 3}. Whether apoA1 binds directly to ABCA1 or to a lipid on the cell surface is controversial and several models have been proposed for this binding¹⁻⁵. ApoA1 can be chemically cross linked to ABCA1⁶; but, purified epitope tagged ABCA1 does not bind to apoA1 in the presence or absence of several classes of phospholipids including PS⁴. Thus, the mechanism by which ABCA1 mediates apoA1 binding and the assembly of nascent HDL is not well characterized. Here we show that apoA1 binds specifically to phosphatidylinositol (4,5) bis-phosphate (PIP2), and that ABCA1 translocates PIP2 to the outer leaflet of the cell membrane. Using specific ABCA1 mutations it was found that the PIP2 translocation of ABCA1 is independent from its PS translocation activity. It was also found that cell surface PIP2 is required to mediate apoA1 binding and cholesterol efflux. Furthermore, it was discovered that PIP2 is effluxed from cells to apoA1, it is associated with HDL in plasma, and PIP2 on HDL is taken up by target cells in an SR-BI dependent manner. While the present invention is not limited to any particular mechanism, and an understanding of the mechanism is not necessary to practice the invention, it is believed that the PIP2 translocase activity of ABCA1 is crucial for cellular binding of apoA1, lipid efflux, and HDL biogenesis, as well as that PIP2 resides on HDL and is effluxed and taken up similar to other HDL lipids.

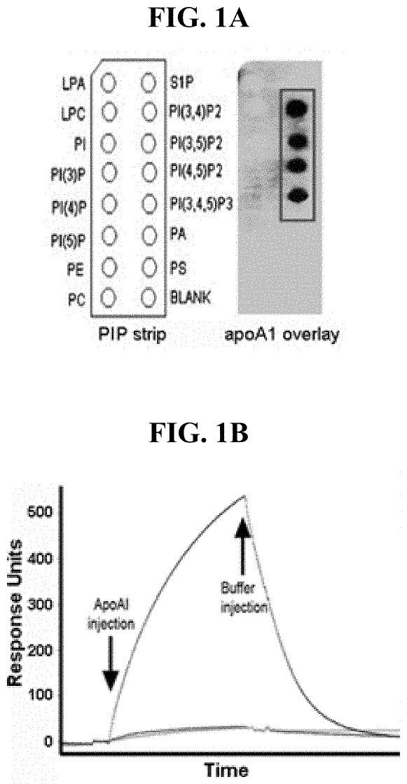

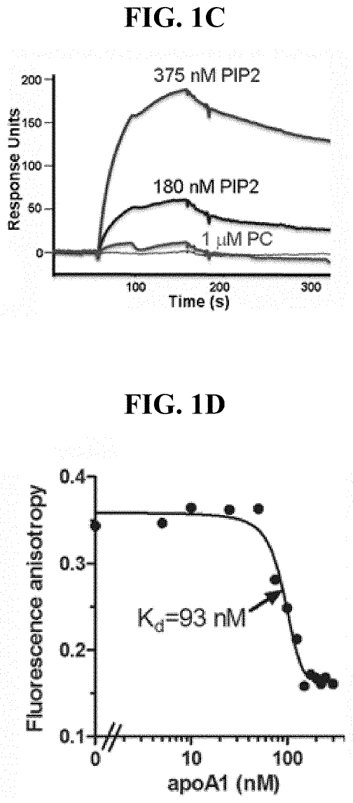

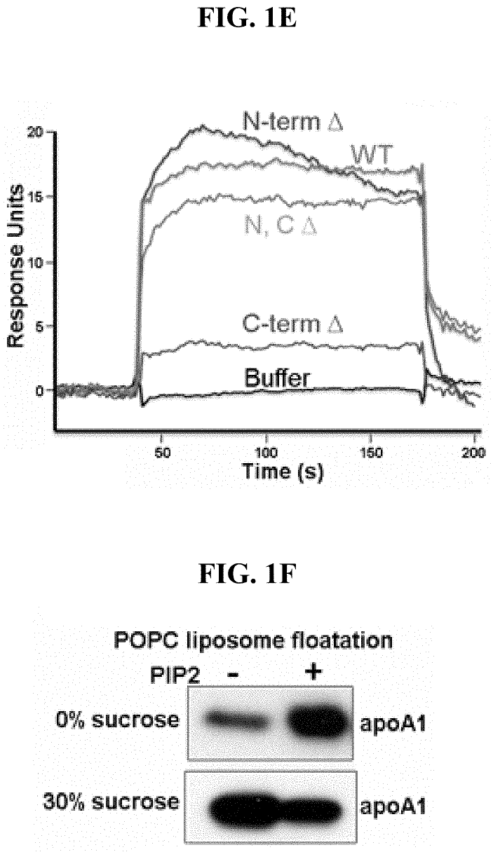

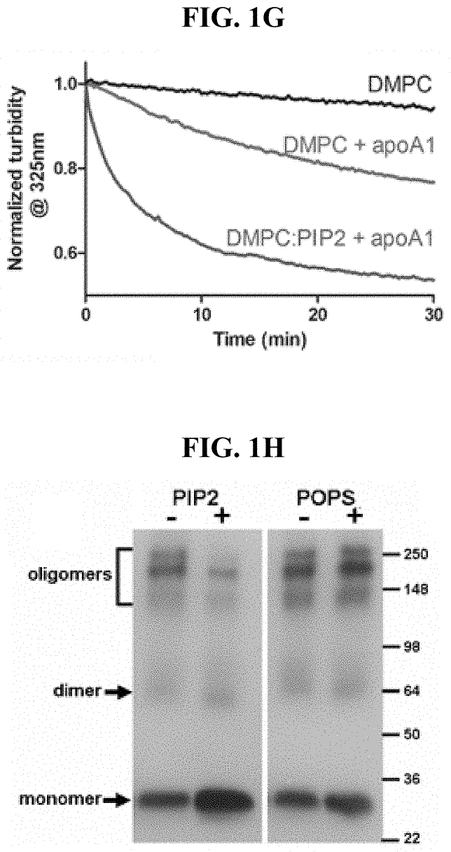

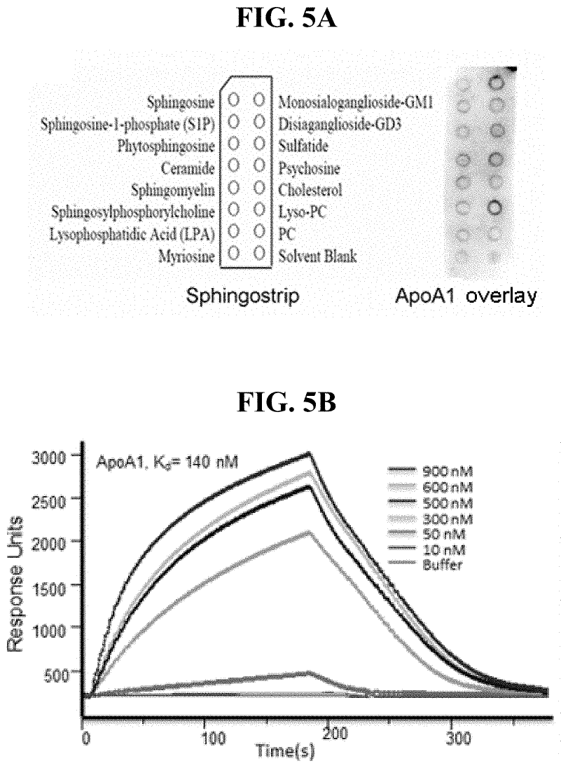

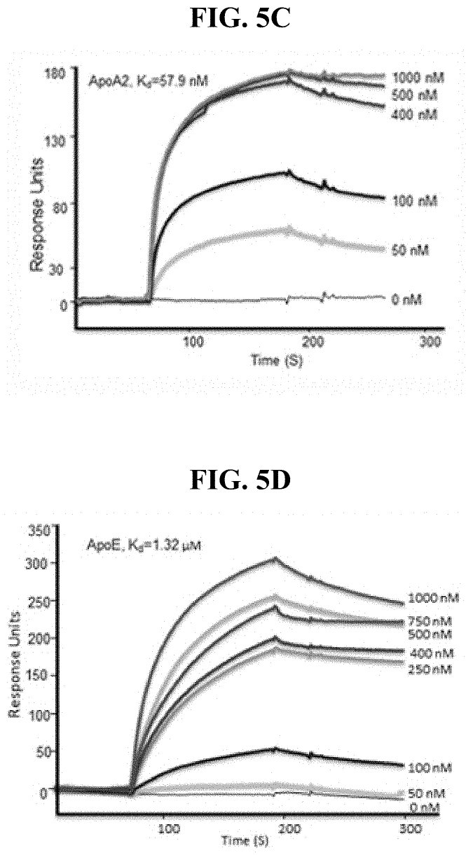

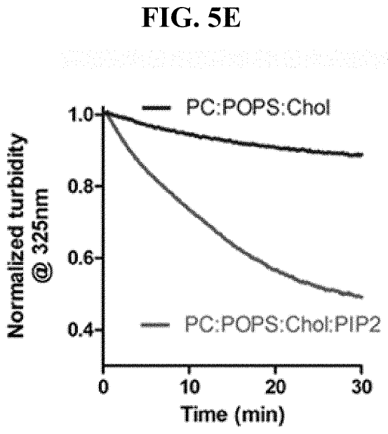

ABCA1 is required for HDL biogenesis. It remodels the plasma membrane, translocating PS to the cell surface, and promoting apoA1 binding. To determine the lipid-binding profile of lipid-free apoA1, lipid-protein overlay assays were performed using phospholipid/phosphatidylinositol phosphate (PIP) and sphingolipid membrane strips. ApoA1 showed direct binding only to PIPs containing 2 or 3 headgroup phosphates and not to other lipids including phosphatidylcholine (PC) or PS (FIG. 1A). Lipid-free apoA1 did not bind to any lipids on the sphingolipid strip, which included sphingosine-1 phosphate, sphingomyelin, ceramide, and cholesterol (FIG. 5A). PIPs can serve as ligands to recruit various proteins to specific membranes, often via their pleckstrin homology (PH) domains. Thus, PIPs are important in vesicle trafficking, co-localization of proteins on membranes, and PIP2 can serve as a precursor for the second messenger inositol triphosphate⁷.

Since PI(4,5)P2 is a major cellular PIP species that is particularly enriched at the cell surface^{8, 9}, further experiments were performed using this PIP2 species. Binding of apoA1 to immobilized PIP2 was demonstrated by surface plasmon resonance (SPR) (FIG. 1B). In addition, PIP2, but not PC, showed direct binding to immobilized apoA1 in dose-dependent manner (FIG. 1C). In solution studies, fluorescence anisotropy demonstrated high affinity binding of apoA1 to Bodipy-fatty acid labeled PIP2 (k_{d}=93 nM, FIG. 1D). This affinity was similar to that obtained by SPR (FIG. 5B). Different truncation mutants of apoA1 were probed to determine the domain that binds to PIP2. The wild type (full length), N-terminal deleted (1-434), and N- and C-terminal double deleted (43-185 AA) apoA1 isoforms retain ABCA1-dependent cholesterol acceptor activity, but the C-terminal deleted isoform (190-2434) is defective in this activity 10, 11.

It was found that all of the efflux competent apoA1 isoforms were capable of binding to PIP2 in an SPR study, but that the C-terminal deleted isoform was not able to bind to PIP2, mirroring its defective efflux acceptor activity (FIG. 1E). Thus, the central domain of apoA1 was sufficient to mediate PIP2 biding. Many proteins bind PIP2 through their conserved PH domains¹²; however, some proteins bind PIP2 through other domains including a cationic grip domain that binds the PIP2 head group electrostatically 13, 14. ApoA1 does not contain a PH domain, but its class A amphipathic helical structure contains a surface lined with positively charged lysine and arginine residues, which, not necessary to understand or practice the present invention, is postulated to be responsible for its PIP2 binding activity. In support of this hypothesis, apoA2 and apoE, other ABCA1 acceptors with similar class A amphipathic helical structures, also showed direct binding to PIP2 via SPR (FIG. 5C, 5D).

apoA1 binding to PIP2 in a lipid environment was confirmed via a liposome floatation assay. ApoA1 was added to palmitoyloleoyl-phosphatidylcholine (POPC) liposomes with or without PIP2 (5 mole %) in 30% sucrose, and after step-gradient ultracentrifugation it was observed increased co-migration of apoA1 with the PIP2 liposomes vs. control liposomes in the top 0% sucrose gradient fraction (FIG. 1F). To determine the consequence of apoA1 binding to liposomes containing PIP2, dimyristyl-phosphatidylcholine (DMPC) multilamellar vesicles (MLV) were prepared with or without PIP2 (5 mole %) and a clearance assay was performed¹⁵.

The addition of lipid-free apoA1 solubilized the PIP2 containing MLVs much faster and to a greater extent than the DMPC-only MLVs (FIG. 1G). Furthermore, the addition of 5% PIP2 to MLVs made from POPC:cholesterol:PS (70:20:10) allowed apoA1 to solubilize these MLVs (FIG. 5E), which was performed at pH 5 where these MLVs have increased reactivity to apoA1¹⁶. Thus, in several cell-free systems apoA1 binds to PIP2 which can lead to increased lipid solubilization. Lipid-free apoA1 exists in equilibrium between its monomeric and oligomeric forms, and the lipid-free monomer is postulated to mediate the initial interaction with the cell membrane and act as the primary ABCA1 acceptor¹⁷. It was found that pre-incubating PIP2, but not PS, with lipid-free apoA1 shifted the equilibrium towards the monomeric form, as assessed by SDS-PAGE after addition of the chemical crosslinker BS3 (FIG. 1H). Thus, PIP2 both recruits apoA1 to the lipid surface and promotes its monomeric structure, favored for lipid solubilization.

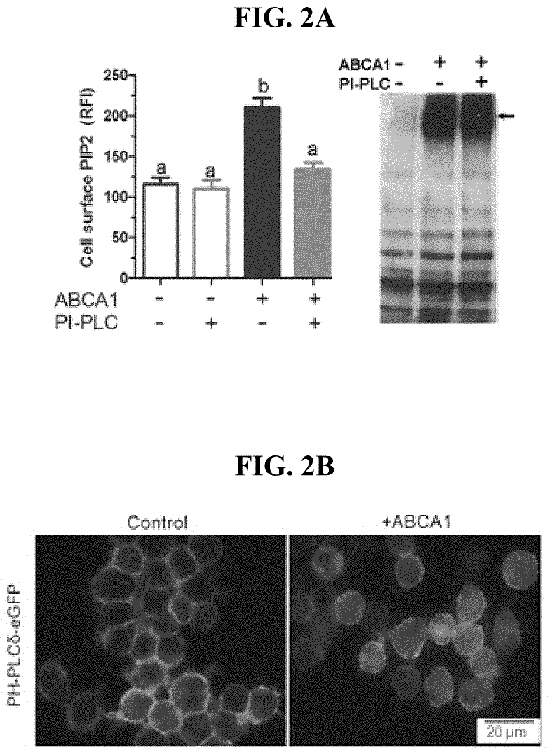

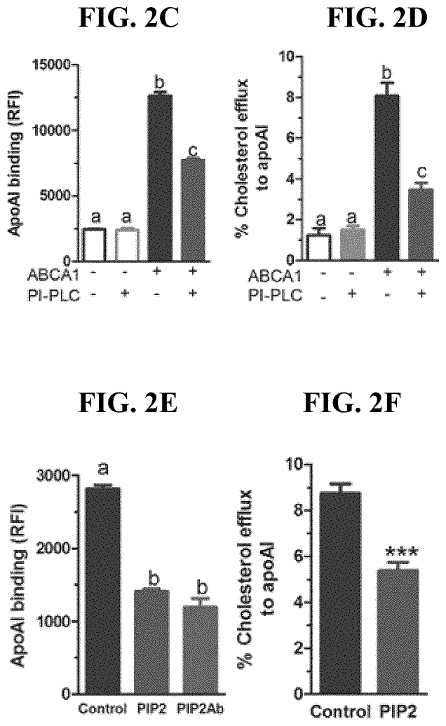

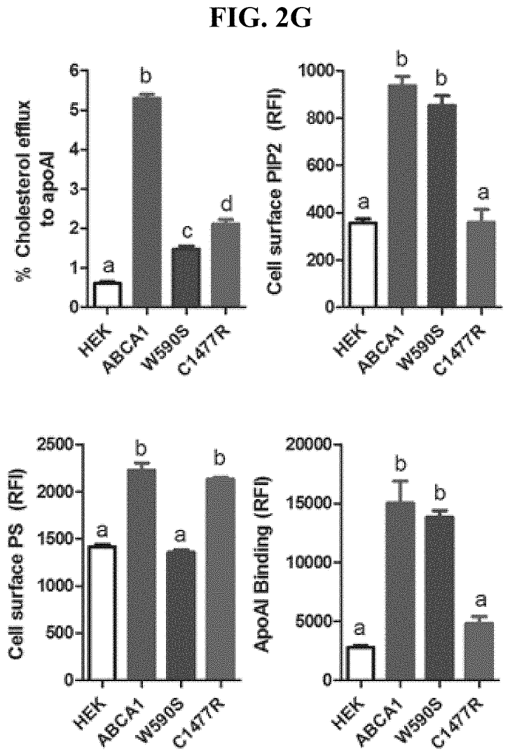

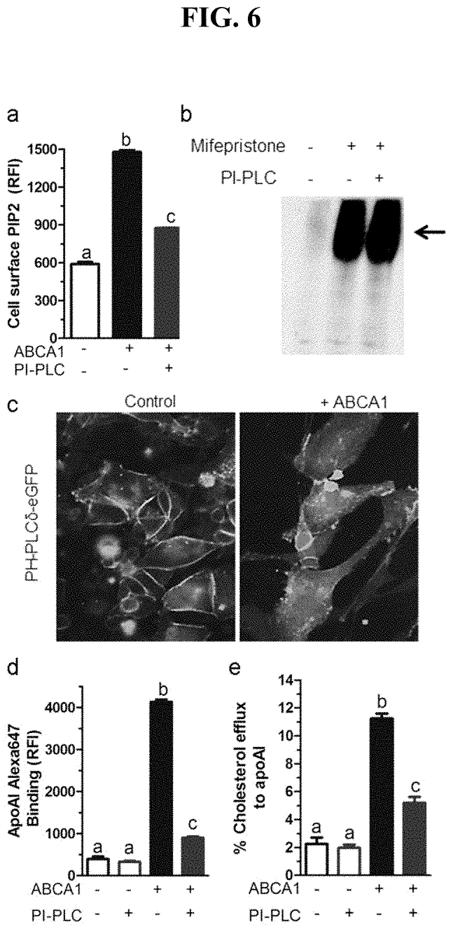

PIP2 is thought to be localized at the inner leaflet of plasma membrane where it plays important roles in targeting proteins to the membrane, membrane trafficking, and signal transduction^{18, 19}. Since ABCA1 has well defined PS outward translocase (floppase) activity³, the possibility was considered that ABCA1 might act as a PIP2 floppase as well. Increased levels of cell surface PIP2 were detected in RAW264.7 cells (FIG. 2A) and stably transfected ABCA1-inducible BHK cells²⁰ (FIG. 6 Panel a) after induction of ABCA1 by 8Br-cAMP or mifepristone, respectively. These enhanced levels of cell surface PIP2 could be catabolized by treatment with exogenous phosphatidylinositol specific phospholipase C (PI-PLC) (FIG. 2A, FIG. 6 Panel a). PI-PLC treatment had no effect on ABCA1 expression in either cell line (FIG. 2A, FIG. 6 Panel b). To confirm the role of ABCA1 in translocating PIP2 to the cell surface, cells were stably transfected with a PIP2-binding reporter protein (2X-PH-PLCδ-eGFP) that does not bind to other PIP species²¹. This reporter was localized mainly to the plasma membrane in untreated RAW264.7 and BHK cells, consistent with PIP2 localization in the inner leaflet of the membrane; however, upon ABCA1 induction, the PIP2 reporter redistributed with less prominent plasma membrane, and increased cytosolic, localization (FIG. 2B, FIG. 6 Panel c), which was attribute to PIP2 translocation to the outer leaflet of plasma membrane. Thus, in addition to the well-known exposure of cell surface PS by ABCA1, cell surface remodeling with increased PIP2 exposure was demonstrated.

To probe the consequences of the ABCA1-mediated increase in cell surface PIP2, the effect of PI-PLC treatment on apoA1 binding and cholesterol efflux was determined. In both RAW264.7 and stably transfected HEK293 cells, PI-PLC treatment greatly diminished ABCA1-induced apoA1 binding (FIG. 2C, FIG. 6 Panel d). Additionally, PI-PLC treatment greatly diminished ABCA1 mediated cholesterol efflux to apoA1 from RAW264.7 and ABCA1-inducible BHK cells (FIG. 2D, FIG. 6 Panel e). Blocking of exposed PIP2 with an anti-PIP2 monoclonal antibody led to decreased apoA1 binding to the cell surface (FIG. 2E), confirming that PIP2 plays a role in apoA1 binding. Pre-incubation of apoA1 with PIP2 decreased apoA1 binding and cholesterol efflux in ABCA1-induced RAW264.7 cells (FIG. 2E, f). Thus, exogenous PIP2 bound to apoA1 competed against cell surface PIP2.

The PS floppase and apoA1 cellular binding activities of ABCA1 can be distinguished from each other using naturally occurring Tangier disease-associated mutations in the first and second large extracellular domains of ABCA1^{2, 22-24.} Cells expressing the W590S ABCA1 isoform are deficient in PS floppase activity but display normal apoA1 binding activity, while cells expressing the C1477R ABCA1 isoform have normal PS floppase activity but are deficient in apoA1 binding. To evaluate if the PS and PIP2 floppase activities of ABCA1 are independent of each other, stably transfected HEK293 cells with equal expression of WT-ABCA1-GFP, W590S-ABCA1-GFP, or C1477R-ABCA1-GFP GFP²² were analyzed for cholesterol efflux, cell surface exposure of PS and PIP2, as well as apoA1 binding (FIG. 2G). Cells expressing WT ABCA1 had all of these activities induced vs. control HEK cells. Cells expressing W590S-ABCA1 had defective cholesterol efflux and PS exposure but had normal PIP2 exposure and apoA1 binding activity, while cells expressing C1477R-ABCA1 had defective cholesterol efflux, apoA1 binding, and PIP2 exposure, but had normal PS exposure. While the present invention is not limited to any particular mechanism, and an understanding of the mechanism is not necessary to practice the invention, it was concluded that first large extracellular domain of ABCA1 mediates PS floppase, which remodels the plasma membrane and increases cholesterol extractability²⁵, while the second large extracellular domain of ABCA1 mediates PIP2 floppase, which is required for apoA1 binding. Thus, these two phospholipid floppase activities of ABCA1 are independent of each other and mediated by distinct domains.

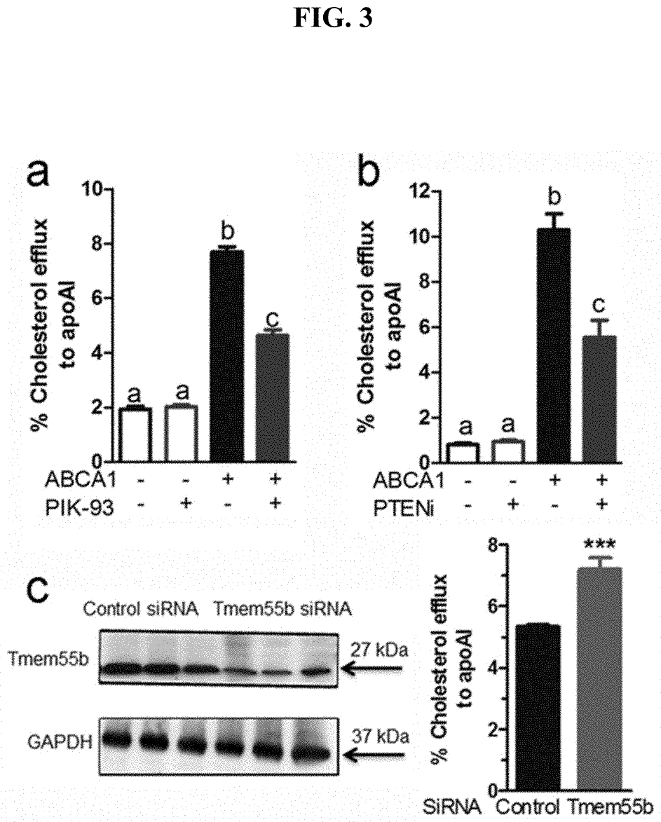

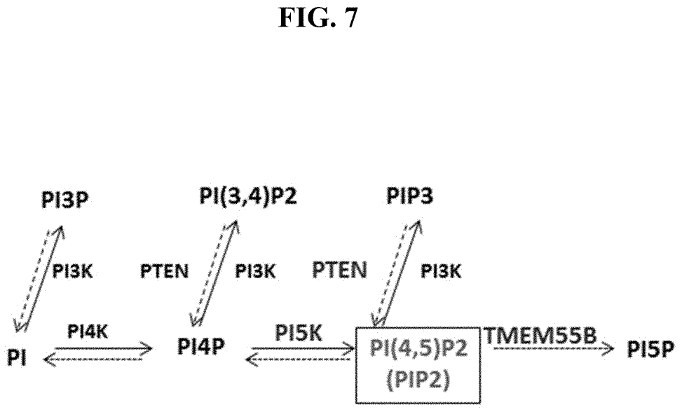

Cellular PIP2 can be generated through de novo phosphorylation of PI4P by PI4P-5 kinase, or via dephosphorylation of PIP3 by PTEN; and, PIP2 can be depleted by the phosphatase activity of Tmem55b^{26,27} (FIG. 7). Treatment of RAW264.7 cells to decrease cellular PIP2 by either PIK-93, a PI4P-5 kinase inhibitor, or SF1670, a PTEN inhibitor, decreased ABCA1-dependent cholesterol efflux to apoA1 (FIG. 3 Panels a, b). Conversely, increasing PIP2 via siRNA mediated knockdown of Tmem55b in RAW264.7 macrophages increased cholesterol efflux to apoA1 (FIG. 3 Panel c). Combined, these studies demonstrate that manipulation of cellular PIP2 levels can modulate ABCA1-mediated cholesterol efflux.

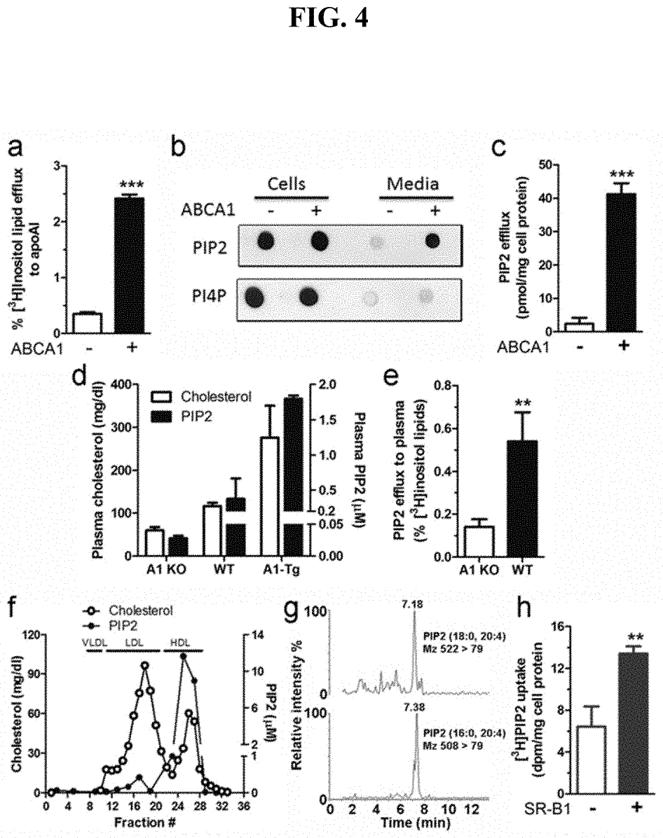

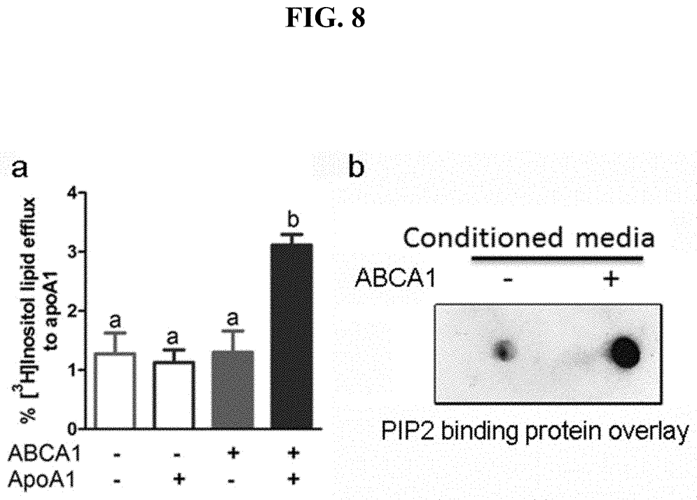

To determine if PIP2 could be effluxed from cells along with other phospholipids and cholesterol during HDL biogenesis cells were labeled with [³H]myo-inositol, and after chasing with apoA1, the conditioned media radioactivity in extracted lipids was measured. Efflux of inositol labeled lipids was increased upon ABCA1 induction in both RAW264.7 and BHK cells (FIG. 4 Panel a, FIG. 8 Panel a). However, the inositol lipid fraction can contain phosphatidylinositol (PI) and any of the PIP species. Thus, a protein-lipid overlay assay was performed of lipids extracted from apoA1-containing conditioned media derived from cells with or without ABCA1 expression; and, the presence of PIP2 or PI4P was detected using tagged PIP2 and PI4P binding proteins, respectively.

The conditioned media obtained from RAW264.7 and BHK cells contained elevated PIP2 only in the ABCA1-induced cells (FIG. 4 Panel b, FIG. 8 Panel b). In contrast, PI4P in the conditioned media was not increased by ABCA1 induction in RAW264.7 cells (FIG. 4 Panel b). An ELISA assay was used to quantify the amount of PIP2 in the conditioned media. RAW264.7 cells expressing ABCA1 effluxed ˜20-fold more PIP2 to apoA1 vs. control cells (FIG. 4 Panel c). Plasma from apoA1 knockout (A1 KO), wild type (WT), and human apoA1 transgenic (A1-Tg) mice contained apoA1-gene dosage dependent levels of both cholesterol and PIP2, with 64-fold higher PIP2 levels in the A1-Tg vs. A1 KO mice (FIG. 4 Panel d). WT mice had plasma levels of ˜0.4 μM PIP2. The low level of plasma PIP2 in A1 KO plasma (˜0.03 μM) implies that most PIP2 is carried on HDL and not complexed with albumin or found free in the plasma.

To determine if PIP2 can be reverse transported from macrophages to the plasma, a modified reverse cholesterol transport study was performed, where macrophages were labeled in culture with [³H]myo-inositol and implanted s.c. into A1 KO and WT mice. Plasma was collected 3 days post implantation, and radioactivity in PIP2 was determined after pulldown with a tagged PIP2 binding protein. Labeled PIP2 was recovered in the plasma, with a higher % of the injected radioactivity found in the WT hosts (FIG. 4 Panel e). FPLC separation of human plasma determined that almost all of the PIP2 was found in the HDL fractions (FIG. 4 Panel f). In human HDL, two PIP2 species containing either 18:0, 20:4 fatty acids or 16:0, 20:4 fatty acids were detected by liquid chromatography tandem mass spectrometry (FIG. 4 Panel g). Therefore, PIP2 is effluxed from cells and is carried on HDL, implying that HDL may serve as a vehicle to deliver PIP2 to target tissues. SR-BI-inducible BHK cells exhibited 2-fold higher uptake of [³H]PIP2 after SR-BI induction (FIG. 4 Panel h), indicating that HDL can deliver PIP2 to target cells.

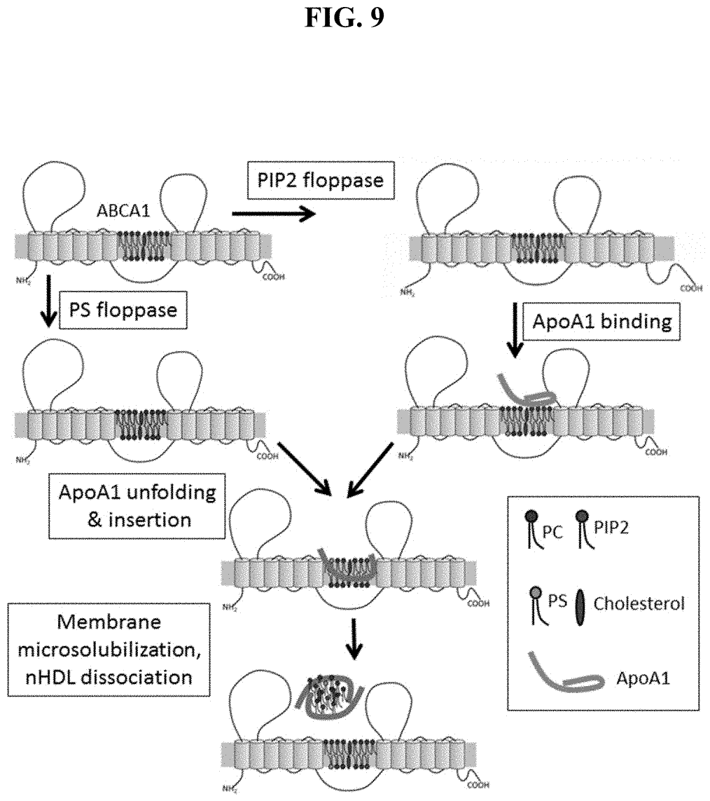

Several models have been proposed for the mechanism of apoA1 binding to ABCA1 expressing cells that initiates nascent HDL assembly: 1) direct interaction between apoA1 and ABCA1; 2) low affinity interaction of apoA1 with ABCA1 followed by high affinity interaction with membrane lipids; 3) ApoA1 interaction with highly curved membrane protrusions caused by the PC floppase activity of ABCA1; and 4) ApoA1 binding to cell surface PS due to the PS floppase activity of ABCA1^{5, 28}. Here, it is demonstrated that apoA1 binding to ABCA1 expressing cells is mediated by the PIP2 floppase activity of ABCA1, and this was put into context in a model for nascent HDL formation (FIG. 9).

The PS floppase activity, mediated by the first large extracellular domain, promotes membrane remodeling that makes the membrane more susceptible to detergents such as sodium, taurocholate or amphipathic proteins such as apoA1^{22,24,25} The PIP2 floppase activity mediated by the second large extracellular domain, promotes apoA1 binding to the cell surface. Once bound to the cell, the PIP2-apoA1 interaction favors apoA1 monomerization that is thought to promote its insertion into the membrane¹⁷. It was previously demonstrated that ABCA1-mediated cellular binding of apoA1 promotes the partial unfolding of the apoA1 N-terminal helical hairpin on the cell surface²². This unfolded apoA1 can then insert into the cell membrane where it can microsolubilize cellular lipids and assemble them into nascent HDL that is released from the cell. Thus, both PS and PIP2 floppase activities are required for maximal cholesterol efflux. ApoA1 is the most abundant apolipoprotein in plasma with normal levels of 1-2 mg/ml. Any weak detergent activity of apoA1 could be detrimental to the host. While the present invention is not limited to any particular mechanism, and an understanding of the mechanism is not necessary to practice the invention, it is speculated that the ABCA1 PIP2 floppase activity may have co-evolved with PIP2 binding activity of apoA1 as a mechanism to prevent the promiscuous detergent activity of apoA1, allowing apoA1 to solubilize lipids from cells under tight control by ABCA1 expression. In addition, the discovery of circulating PIP2 on HDL and its delivery to target cells may open up a new area of HDL-mediated signal transduction that might explain many of the pleiotropic effects of HDL on various cell types.

### Methods:

Materials: PIP strips (P-6001), Sphingo strips (S-6000), PIK-93 inhibitor (B0306), PTEN inhibitor SF1670 (B-0350), PI (4,5)P2 (P-4524), PI (4,5)P2 ELISA kit (K-4500), PI (4)P Grip (G0402), PI (4,5)P2 Grip (G4501), biotin-PIP2 (C-45B6), fatty acid labeled-bodipy PIP2 (C-45F16a), and FITC conjugated Anti-PIP2 antibody (Z-G045) were from Echelon Biosciences. HRP-conjugated GST antibody was from Sigma. Alexa647-Antibody labeling kit was from Molecular Probes (Cat No. A-20186). Purified recombinant human proteins apoA2 (TP721104) and apoE (TP723016) were from Origene. [³H]-labeled PIP2 (NET895005UC), myo-inositol (NET1177001MC), and cholesterol (NET13900) were from Perkin Elmer. ApoA1 was purified form human plasma²⁹, and dialyzed against PBS. Recombinant human apoA1 and truncation mutations were prepared as previously described³⁰. RAW264.7 cells were from ATCC. Mifepristone ABCA1-inducible BHK cells, as previously described³¹ were obtained from Chongren Tang, University of Washington. Mifepristone SR-BI-inducible BHK cells, as previously described³², were obtained from Alan Remaley, NIH. ABCA1-GFP and the mutant isoform stably transfected HEK cells were as previously described²².

Protein-lipid overlay assays: The PIP strip and sphingo strip membranes were blocked with 5% milk powder in PBS-Tween for 30 min, and apoA1 was added at 50 μg/ml and incubated at room temperature for 2 hr. The bound protein was detected by using anti human apoA1 goat (Meridian Life Science, #K45252G) antibody and HRP conjugated anti-goat antibody. HRP was visualized using ECL reagent (Pierce) and exposure to x-ray film. Lipids extracted from conditioned media or cells were dissolved in methanol:chloroform:12N HCl (40:80:1) and spotted onto nitrocellulose membranes. After treating with casein blocker (Thermo scientific; #37528), the membranes were incubated with GST-PLCδ-PH (1 μg/ml, Echelon Biosciences) to detect PIP2, or with GST-SiDC-3C (1 μg/ml, Echelon Biosciences) to detect PI4P. The binding interactions were detected using HRP-conjugated anti-GST antibody (Sigma) and ECL chemiluminescence.

Surface Plasmon resonance: Binding kinetic of PIP2 with different apolipoproteins was analyzed using a Biacore3000 instrument. Either biotinylated apoA1 or biotinylated PIP2 was immobilized on a streptavidin (SA) sensor chip (GE Healthcare). The immobilized apoA1 or PIP2 was stable over the course of the experiment and baseline drift was <10 response units (RU)/h after the washing with Hepes buffered saline (FIBS) buffer. Different concentrations of apoA1 or PIP2 were injected using the KINJECT procedure at flow-rate of 10 μl/min and dissociation was monitored by injecting FIBS buffer. The injections were performed in triplicate for each ligand concentration. For comparing binding kinetics of PIP2 with apoA1, apoA2 and apoE, these proteins were immobilized by covalent coupling on a CMS sensor chip (GE Healthcare) using EDC-NHS reagents. PIP2 was injected as described above. Corrected response data were fitted with BIAevaluation software version 4.01, and K_{d} values were calculated.

Fluorescence anisotropy: Increasing concentrations of apoA1 were incubated with 100 nM fatty acid-labeled bodipy PIP2 in a quartz cuvette at 25° C. Relative anisotropy was determined using polarized filters with excitation at 503 nm and emission at 513 nm in a Perkin Elmer spectrofluorimeter. The K_{d} was determined as the EC50 by non-linear regression of the log apoA1 concentration. A similar K_{d} value was obtained using 400 nM PIP2.

Liposome clearance assay: 1,2-Dimyristoyl-sn-glycero-3-phos-phocholine (DMPC) or 1-palmitoyl-2-oleoyl-sn-glycero-3-phosphocholine (POPC) (Avanti Polar Lipids) with or without 5% PIP2 were dissolved in chloroform: methanol (2:1 v/v) and were dried in a stream of nitrogen and placed in vacuum overnight. DMPC or POPC was rehydrated in PBS by five cycles of freeze-thaw and extensive vortexing to form multilamellar vesicles (MLVs) at 5 mg/ml. These MLVs were subjected to apoA1 solubilization assay. Briefly, the MLVs dissolved in Tris-buffered saline-EDTA (pH7.5) were incubated with human apoA1 at 25° C. MLV solubilization by human apoA1 was monitored by measuring sample turbidity (absorbance) at 325 nm using a plate reader.

Liposome floatation assay. POPC MLVs made with or without 5 mole % PIP2 were incubated at room temperature with apoA1 (20:1, lipid:apoA1 mass ratio) in 30% sucrose and placed at bottom of a sucrose density step gradient and subjected to ultracentrifugation, as previously described³³. Equal volume aliquots of the top (0% sucrose) and bottom (30% sucrose) fractions were precipitated and analyzed by SDS-PAGE and apoA1 western blot.

ApoA1 cross linking: ApoA1 was incubated in the presence or absence of PIP2 or POPC at 1:1 mole ratios and then incubated with bis(sulfosuccinimidyl) suberate (BS3, Pierce) crosslinker at room temperature for 30 minutes. The reactions were quenched with 1M Tris, pH 8.0 and samples were analyzed by SDS-PAGE and apoA1 western blot.

Cell growth and ABCA1 induction: All cell culture incubations were performed at 37° C. in humidified 5% CO₂ incubator. The growth media was Dulbecco's Modified Eagle Medium (DMEM) with 10% fetal calf serum, 100 μg/mL penicillin, 100 μg/mL streptavidin. ABCA1 was induced in RAW26.47 cells by 16-24 hr incubation with 0.3 mM 8Br-cAMP³⁴. ABCA1 was induced in BHK cells by 16-24 hr incubation with 10 nM mifepristone³¹. Inducers were included in the media during subsequent assays. ABCA1 expression was confirmed by western blot using the AC10 antibody (Santa Cruz Biotech).

Cholesterol efflux assay: On day 1, cells were plated on 24-well plates at a density of 20,000 to 400,000 cells per well. On day 2, the cells were labeled with 0.5 Ki/ml [³H]cholesterol in DMEM containing 1% FBS. On day 3, the cells when indicated were treated with or without ABCA1 inducers in serum-free DMEM. On day 4 (or day 3 for HEK293 cells and ABCA1 stably transfected cells) the cells were washed and chased for 4-6 hr in serum-free DMEM in the presence or absence of 5 μg/ml apoA1. The radioactivity in the chase media was determined after brief centrifugation to pellet any residual debris. Radioactivity in the cells was determined by extraction in hexane:isopropanol (3:2) with the solvent evaporated in a scintillation vial prior to counting. The percent cholesterol efflux was calculated as 100×(medium dpm)/(medium dpm+cell dpm).

Inositol lipid efflux: For [³H]myo-inositol labeling, the growth medium was replaced with inositol-free DMEM (including 10% fetal calf serum, 100 μg/mL penicillin, 100 μg/mL streptavidin and 2 mM glutamine) and [³H]myo-inositol was added to a final concentration of 40 μCi/mL for 24 hr followed by ABCA1 induction in serum-free DMEM where indicated. The cells were washed and chased for 4-6 hr in serum-free medium in the presence or absence of 5 μg/ml apoA1. The chase media was collected, centrifuged to remove any cell debris, and acidic lipid fractions containing PIPs were isolated as following the protocol provided by Echelon Bioscience: 1 ml medium was resuspended in 750 μL chloroform/methanol/12N HCl (40:80:1, v/v/v) and incubated for 15 min at RT while vortexing the sample for 1 min every 5 min. After transferring the tube to ice, 250 μL cold chloroform and 450 μL cold 0.1 M HCl was added followed by 1 min vortexing and centrifugation (6,500×g, 2 min at 4° C.). The bottom organic phase was transferred to a fresh tube, dried under N₂ gas in a scintillation vial and subjected to scintillation counting.

Inositol lipid reverse transport in vivo. Bone-marrow derived macrophages from C57BL/6 mice were labeled with 40 μci/ml of [³H]myo-inositol for 24 h as described above. An aliquot of the cells was extracted in hexane:isopropanol (3:2) to determine total ³H dpm in inositol labeled lipids. ˜1.8×10⁶ dpm of labeled macrophages were injected s.c. into the back of each mouse. 3 days later, plasma was collected, followed by acidic extraction of lipids, resupended in PBS-PS (PBS 0.25% Protein Stabilizer Echelon #K-GS01). This was incubated with PH-PLC δ-GST tagged protein (Echelon). The PIP2 bound to GST tagged protein was separated from other inositol labeled lipids by incubation with glutathione-beads, and after washing the bound PIP2-protein complex was eluted by incubation with 50 mM Tris, 10 mM reduced glutathione, pH=8.0. The eluate was subjected to scintillation counting. The % efflux to plasma was determined by calculating 100×PIP2 dpm calculated in total body plasma divided by the injected inositol lipid dpm.

PIP2 cellular reporter assay: RAW264.7 macrophages and ABCA1-inducible BHK cells were transfected with 2PH-PLCδ-GFP plasmid (Addgene) using Lipofectamine 2000 transfection reagent (ThermoFisher Scientific). The GFP positive colonies were visually identified by epifluorescent microscopy selected and expanded in 1.5 mg/ml G418. RAW264.7 cells and BHK cells were induced to express ABCA1 as indicated. The cells were washed with PBS and visualized by epifluorescent microscopy. Images were taken using the same exposure time.

Tmem55b knockdown: The siRNA to mouse Tmem55b (Origene, #SR408149) and scrambled control were transfected in RAW264.7 cells using siTran 1.0 (Origene). The cellular protein extracts were prepared using NP-40 lysis buffer containing protease inhibitors. The knockdown efficacy was determined by western blot using anti Tmem55b antibody (Santa Cruz).

Cell surface PS, PIP2, and apoA1 binding assays via flow cytometry: Cell surface PS levels were determined by flow cytometry after cell scraping in PBS, re-suspension in Annexin V binding buffer, and incubation with AnnexinV-Cy5 (Biovision) at room temperature for 5 minutes in the dark. Cell surface PIP2 levels were determined by flow cytometry by incubation with Alexa647 or FITC labeled anti-PIP2 antibody (Echelon) in phenol red-free, serum-free, DMEM at room temperature for 30 min. Human apoA1 was labeled with Alexa647 (Molecular Probes) on free amines using a 6:1 mole ratio of dye:apoA1. Alexa647-apoA1 binding was determined by flow cytometry after incubation with cells for 45 minutes at room temperature. All flow cytometry assays were performed on a BD Biosciences LSRFortessa cytometer using the following settings: FITC, Ex: 488 nm, Em:505-525 nm (Filter 515/20); Cy5 and Alexa 647, Ex: 639 nm, Em: 650-670 nm (Filter 660/20). Data was analyzed by Flowjo software and the median relative fluorescent intensities were compared.

PIP2 ELISA: PIP2 was quantified by using the PI(4,5)P2 Mass ELISA kit from Echelon Biosciences, following the protocol provided. Briefly, conditioned media or plasma was extracted using the acidic lipid extraction protocol described above, dried, and resuspended in PBS-PS. Cells were suspended, pelleted, and washed in cold 5% TCA with 1 mM EDTA. Cell neutral lipids were extracted in 1 mL chloroform:methanol (1:2). The pellet containing acidic lipids was extracted in 750 μL chloroform:methanol:12N HCl (40:80:1). 250 μL cold chloroform and 450 μL cold 0.1 M HCl was added to the supernatant. The bottom organic phase was dried, suspended in PBS-PS. Media and cell extracts in PBS-PS were subjected to the PIP2 Mass ELISA assay according the Echelon protocol

Plasma analyses: 0.5 ml of human plasma (obtained under informed consent in an IRB approved protocol) was separated by fast protein liquid chromatography (FPLC) on a Superose 6 column (Amersham), and 0.5 ml fractions were collected. Total cholesterol was measured in mouse plasma or human FPLC fractions using the Cholesterol LiquiColor kit (Stanbio Laboratory). PIP2 concentration was determined using the PIP2 ELISA assay (described above). Human HDL was isolated by equilibrium density ultracentrifugation at density between 1.063 and 1.21 g/ml. LC-MS/MS was used for PIP2 profiling in human HDL as previously described³⁵. In brief, HDL lipids extracts were rapidly dried under nitrogen flow, suspended in 200 μl methanol/water (70:30), and stored under an argon atmosphere at −20° C. until analysis within 24 hr. 20 μl of the extract was introduced onto a 2690 HPLC system (Waters, Milford, Mass.) and phospholipids were separated through a C18 column (2×50 mm, Gemini 5 Phenomenex, Rancho Palos Verdes, Calif.) under gradient conditions at flow rate of 0.3 ml/min. A gradient was used by mixing mobile phase A (Methanol/water (70:30) containing 0.058% ammonium hydroxide) and B (acetonitrile/2-propanol (50:50) containing 0.058% ammonium hydroxide) as follows: isocratic elution with 100% A for 1 min, linear gradient to 100% B from 1 to 6 min, kept at 100% B for 10 min and then equilibrated with 100% A for 7 min. The HPLC column effluent was introduced onto a triple quadruple mass spectrometer (Quattro Ultima Micromass, Beverly, Mass.) and analyzed at negative electrospray ionization in the multiple reaction monitoring (MRM) mode for the targeted PIP2. The MRM transitions used to detect the PIP2 was the mass to charge ratio (m/z) for the molecular anion [MH]⁻ and the product ion at m/z 79, arising from its phosphate group (i.e. [MH]⁻→m/z 79).

SR-BI mediated PIP2 uptake: Mifepristone SR-BI-inducible BHK cells were treated with 10 nM mifepristone to for 14 hr. 0.5 μCi [³H] PIP2 was dried down and 650 μg (protein) of human HDL was added and incubated for 6 hr at room temperature to absorb PIP2 into HDL. The radiolabeled PIP2-HDL complex at 100 μg/ml final concentration was incubated with cells in serum free media for 4 hr at 37° C. Cellular lipids were extracted and ³H was determined by scintillation counting, and normalized to cellular protein after lysis in 0.2 N NaOH, 0.2% SDS.

Statistical analyses: Data are shown as mean±SD. Comparisons of 2 groups were performed by a 2-tailed t test, and comparisons of 3 or more groups were performed by ANOVA with Bonferroni posttest. All statistics were performed using Prism software (GraphPad).

### REFERENCES

1. Wang, N., Silver, D. L., Thiele, C. & Tall, A. R. ATP-binding cassette transporter A1 (ABCA1) functions as a cholesterol efflux regulatory protein. J. Biol. Chem. 276, 23742-23747 (2001).
2. Fitzgerald, M. L. et al. Naturally occurring mutations in the largest extracellular loops of ABCA1 can disrupt its direct interaction with apolipoprotein A-I. J. Biol. Chem. 277, 33178-33187 (2002).
3. Alder-Baerens, N. et al. Headgroup-specific exposure of phospholipids in ABCA1-expressing cells. J. Biol. Chem. 280, 26321-26329 (2005).
4. Reboul, E., Dyka, F. M., Quazi, F. & Molday, R. S. Cholesterol transport via ABCA1: new insights from solid-phase binding assay. Biochimie 95, 957-961 (2013).
5. Phillips, M. C. Molecular mechanisms of cellular cholesterol efflux. J. Biol. Chem. 289, 24020-24029 (2014).
6. Wang, N. et al. A PEST sequence in ABCA1 regulates degradation by calpain protease and stabilization of ABCA1 by apoA-I. J. Clin. Invest. 111, 99-107 (2003).
7. Sun, Y., Thapa, N., Hedman, A. C. & Anderson, R. A. Phosphatidylinositol 4,5-bisphosphate: targeted production and signaling. BioEssay: 35, 513-522 (2013).
8. Czech, M. P. PIP2 and PIP3: complex roles at the cell surface. Cell 100, 603-606 (2000).
9. Le Roy, C. & Wrana, J. L. Clathrin- and non-clathrin-mediated endocytic regulation of cell signalling. Nature reviews. Mol. Cell Biol. 6, 112-126 (2005).
10. Vedhachalam, C. et al. ABCA1-induced cell surface binding sites for ApoA-I. Arterioscler, Thromb. Vasc. Biol. 27, 1603-1609 (2007).
11. Davidson, W. S., Hazlett, T., Mantulin, W. W. & Jonas, A. The role of apolipoprotein AI domains in lipid binding. Proc. Natl. Acad. Sci. USA 93, 13605-13610 (1996).
12. Harlan, J. E., Hajduk, P. J., Yoon, H. S. & Fesik, S. W. Pleckstrin homology domains bind to phosphatidylinositol-4,5-bisphosphate. Nature 371, 168-170 (1994).
13. Poon, I. et al. Phosphoinositide-mediated oligomerization of a defensin induces cell lysis. eLife 3, e01808 (2014).
14. Baxter, A. A. et al. The tomato defensin TPP3 binds phosphatidylinositol (4,5)-bisphosphate via a conserved dimeric cationic grip conformation to mediate cell lysis. Mol. Cell Biol. (2015).
15. Durbin, D. M. & Jonas, A. Lipid-free apolipoproteins A-I and A-II promote remodeling of reconstituted high density lipoproteins and alter their reactivity with lecithin:cholesterol acyltransferase. J. Lipid Res. 40, 2293-2302 (1999).
16. Fukuda, M. et al. Conformational change of apolipoprotein A-I and HDL formation from model membranes under intracellular acidic conditions. J. Lipid Res. 49, 2419-2426 (2008).
17. Gursky, O., Jones, M. K., Mei, X., Segrest, J. P. & Atkinson, D. Structural basis for distinct functions of the naturally occurring Cys mutants of human apolipoprotein A-I. J. Lipid Res. 54, 3244-3257 (2013).
18. Lemmon, M. A. Membrane recognition by phospholipid-binding domains. Nature Rev. Mol. Cell Biol. 9, 99-111 (2008).
19. Cantley, L. C. The phosphoinositide 3-kinase pathway. Science 296, 1655-1657 (2002).
20. Oram, J. F., Vaughan, A. M. & Stocker, R. ATP-binding cassette transporter A1 mediates cellular secretion of alpha-tocopherol. J. Biol. Chem. 276, 39898-39902 (2001).
21. Varnai, P. & Balla, T. Visualization of phosphoinositides that bind pleckstrin homology domains: calcium- and agonist-induced dynamic changes and relationship to myo-[3H]inositol-labeled phosphoinositide pools. J. Cell Biol. 143, 501-510 (1998).
22. Wang, S., Gulshan, K., Brubaker, G., Hazen, S. L. & Smith, J. D. ABCA1 Mediates Unfolding of Apolipoprotein AI N Terminus on the Cell Surface Before Lipidation and Release of Nascent High-Density Lipoprotein. Arterioscler, Thromb. Vasc. Biol. 33, 1197-1205 (2013).
23. Tanaka, A. R. et al. Effects of mutations of ABCA1 in the first extracellular domain on subcellular trafficking and ATP binding/hydrolysis. J. Biol. Chem. 278, 8815-8819 (2003).
24. Nagao, K., Zhao, Y., Takahashi, K., Kimura, Y. & Ueda, K. Sodium taurocholate-dependent lipid efflux by ABCA1: effects of W590S mutation on lipid translocation and apolipoprotein A-I dissociation. J. Lipid Res. 50, 1165-1172 (2009).
25. Gulshan, K., Brubaker, G., Wang, S., Hazen, S. L. & Smith, J. D. Sphingomyelin Depletion Impairs Anionic Phospholipid Inward Translocation and Induces Cholesterol Efflux. J. Biol. Chem. (2013).
26. Ungewickell, A. et al. The identification and characterization of two phosphatidylinositol-4,5-bisphosphate 4-phosphatases. Proc. Natl. Acad. Sci. USA 102, 18854-18859 (2005).
27. Medina, M. W. et al. Transmembrane protein 55B is a novel regulator of cellular cholesterol metabolism. Arterioscler, Thromb. Vasc. Biol. 34, 1917-1923 (2014).
28. Wang, S. & Smith, J. D. ABCA1 and nascent HDL biogenesis. Biofactors 40, 547-554 (2014).
29. Brewer, H. B., Jr., Ronan, R., Meng, M. & Bishop, C. Isolation and characterization of apolipoproteins A-I, A-II, and A-IV. Methods Enzymol. 128, 223-246 (1986).
30. Gross, E., Peng, D. Q., Hazen, S. L. & Smith, J. D. A novel folding intermediate state for apolipoprotein A-I: role of the amino and carboxy termini. Biophys. J. 90, 1362-1370 (2006).
31. Vaughan, A. M. & Oram, J. F. ABCA1 redistributes membrane cholesterol independent of apolipoprotein interactions. J. Lipid Res. 44, 1373-1380 (2003).
32. Vickers, K. C., Palmisano, B. T., Shoucri, B. M., Shamburek, R. D. & Remaley, A. T. MicroRNAs are transported in plasma and delivered to recipient cells by high-density lipoproteins. Nature Cell Bio. 13, 423-433 (2011).
33. Manneville, J. B., Leduc, C., Sorre, B. & Drin, G. Studying in vitro membrane curvature recognition by proteins and its role in vesicular trafficking. Methods Cell Biol. 108, 47-71 (2012).
34. Le Goff, W., Zheng, P., Brubaker, G. & Smith, J. D. Identification of the cAMP-responsive enhancer of the murine ABCA1 gene: requirement for CREB1 and STAT3/4 elements. Arterioscler, Thromb. Vasc. Biol. 26, 527-533 (2006).
35. Wenk, M. R. et al. Phosphoinositide profiling in complex lipid mixtures using electrospray ionization mass spectrometry. Nature Biotech. 21, 813-817 (2003).

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein

We claim:

1. A method performing an activity based on concentration level of PIP2 phospholipid in a biological sample from a subject comprising:
a) determining the concentration level of total PIP2 in a biological sample from a subject, and/or determining the concentration level of HDL-associated PIP2 in said biological sample from said subject; and
b) performing at least one of the following:
i) identifying decreased total or HDL-associated PIP2 levels in said biological sample, and treating said subject with a CVD therapeutic agent;
ii) generating and/or transmitting a report that indicates said total or HDL-associated PIP2 levels are decreased in said sample, and that said subject is in need of a CVD therapeutic agent;
iii) generating and/or transmitting a report that indicates said total or HDL-associated PIP2 levels are decreased in said sample, and that said subject has or is at risk of cardiovascular disease or complication of cardiovascular disease;
iv) generating and/or transmitting a report that indicates said total or HDL-associated PIP2 levels are elevated in said sample, and that said subject has increased reverse-cholesterol transport function;
v) characterizing said subject as having CVD or having an increased risk for having or developing CVD.

2. The method of claim 1, wherein said CVD therapeutic agent is selected from the group consisting of: an antibiotic, a probiotic, an alpha-adrenergic blocking drug, an angiotensin-converting enzyme inhibitor, an angiotensin receptor antagonist, an antiarrhythmic drug, an anticoagulant, an antiplatelet drug, a thromybolytic drug, a beta-adrenergic blocking drug, a calcium channel blocker, a brain acting drug, a cholesterol-lowering drug, a digitalis drug, a diuretic, a nitrate, a peripheral adrenergic antagonist, a TMEM55b inhibitor, a OCRL1 inhibitor, and a vasodilator.

3. The method of claim 1, wherein said biological sample is a plasma sample.

4. The method of claim 1, wherein the biological sample is treated to isolate HDL particles, and treating the HDL sample or the unfractionated sample with solvents to extract PIP2 away from proteins in the HDL of unfractionated sample.

5. The method of claim 4, wherein said biological sample is treated with ultracentrifugation or apoB precipitation reagent to generate said HDL purified sample, wherein said HDL purified sample is free of detectable LDL, IDL, and VLDL.

6. The method of claim 4, wherein said the HDL sample or the unfractionated sample is treated with weak detergents to cause PIP2 to dissociate away from HDL or sample proteins.

7. The method of claim 1, wherein said cardiovascular disease or complication of cardiovascular disease is one or more of the following: non-fatal myocardial infarction, stroke, angina pectoris, transient ischemic attacks, congestive heart failure, aortic aneurysm, aortic dissection, and death.

8. The method of claim 1, wherein said risk of cardiovascular disease is a risk of having or developing cardiovascular disease within the ensuing three years.

9. The method of claim 1, wherein said determining comprises contacting said bodily sample with an anti-PIP2 antibody.

10. A method of treatment comprising:
a) identifying a subject as having reduced levels of PIP2, and
b) treating said subject with a CVD therapeutic agent.

11. The method of claim 10, wherein said identifying comprises receiving a report that said subject has reduced levels of PIP2.

12. The method of claim 10, wherein said CVD therapeutic agent comprises a lipid reducing agent.

13. The method of claim 10, wherein said CVD therapeutic agent is selected from the group consisting of: an antibiotic, a probiotic, an alpha-adrenergic blocking drug, an angiotensin-converting enzyme inhibitor, an angiotensin receptor antagonist, an antiarrhythmic drug, an anticoagulant, an antiplatelet drug, a thromybolytic drug, a beta-adrenergic blocking drug, a calcium channel blocker, a brain acting drug, a cholesterol-lowering drug, a digitalis drug, a diuretic, a nitrate, a peripheral adrenergic antagonist, a TMEM55b inhibitor, a OCRL1 inhibitor, and a vasodilator.

14. A method for evaluating the effect of a cardiovascular disease (CVD) therapeutic agent on a subject comprising:
a) determining a first level of PIP2 in a bodily sample taken from a subject prior to administration of a CVD therapeutic agent, and
b) determining a second level of PIP2 in a corresponding bodily fluid taken from said subject following administration of said CVD therapeutic agent.

15. The method of claim 14, wherein an increase in said first level to said second level is indicative of a positive effect of said CVD therapeutic agent on cardiovascular disease in said subject.

16. The method of claim 14, wherein said CVD therapeutic agent is selected from the group consisting of: a lipid reducing agent, an anti-inflammatory agent, an insulin sensitizing agent, an anti-hypertensive agent, an anti-thrombotic agent, an anti-platelet agent, a fibrinolytic agent, a direct thrombin inhibitor, an ACAT inhibitor, a TMEM55b inhibitor, a OCRL1 inhibitor, a CETP inhibitor, and a glycoprotein IIb/IIIa receptor inhibitor.

17. A method comprising: administering a transmembrane protein 55B (Tmem55b) inhibitor and/or an inositol polyphosphate-5-phosphatase (OCRL1) inhibitor to a subject, wherein said subject has, or is suspected of having, cardiovascular disease.

18. The method of claim 17, wherein said Tmem55b inhibitor comprises a Tmem55b siRNA sequence, a Tmem55b antisense sequence, a small molecule, and/or an anti-Tmem55b antibody or antigen binding fragment thereof.

19. The method of claim 17, wherein said OCRL1 inhibitor comprises an OCLR1 siRNA sequence, an OCRL1 antisense sequence, a small molecule, and/or an anti-OCRL1 antibody or antigen binding fragment thereof.

20. The method of claim 17, wherein said Tmem55b inhibitor and/or said OCLR1 inhibitor is administered at a level to increase the PIP2 levels in said subject at least 10%.
